(19) United States
(12) Patent Application Publication
(10) Pub. No.: US 2023/0011678 A1
(43) Pub. Date: Jan. 12, 2023

(54) METHOD FOR PREDICTING PROTEIN-PROTEIN INTERACTION

(71) Applicant: BEIJING BAIDU NETCOM SCIENCE TECHNOLOGY CO., LTD., Beijing (CN)

(72) Inventors: Yang Xue, (US); Zijing Liu, Beijing (CN); Xiaomin Fang, Beijing (CN); Fan Wang, Beijing (CN); Jingzhou He, Beijing (CN)

(73) Assignee: BEIJING BAIDU NETCOM SCIENCE TECHNOLOGY CO., LTD., Beijing (CN)

(21) Appl. No.: 17/935,233

(22) Filed: Sep. 26, 2022

(30) Foreign Application Priority Data
Nov. 26, 2021 (CN) 202111423752.5

(51) Int. Cl. G16B 5/00; G16B 15/20; G16B 20/00; G16B 30/00; G16B 40/00

(52) U.S. Cl. CPC G16B 5/00; G16B 15/20; G16B 20/00; G16B 30/00; G16B 40/00

(57) ABSTRACT

Provided is a method for predicting protein-protein interaction. Also provided are an electronic device and a non-transitory computer readable storage medium.

### CROSS-REFERENCE TO RELATED APPLICATIONS

This application is based on and claims priority to the Chinese Patent Application No. 202111423752.5, filed on Nov. 26, 2021, the entire content of which is incorporated herein by reference.

### FIELD

The present disclosure relates to the technical field of artificial intelligence, in particular to the technical field of natural language processing and deep learning, and in more particular to a method for predicting protein-protein interaction, an electronic device and a non-transitory computer readable storage medium.

### BACKGROUND

Prediction of protein-protein interaction is of a great significance for applications such as vaccine design, antibody drug design, and polypeptide drug design. In the process of predicting the protein-protein interaction, accuracy of representing a protein directly affects a prediction results of the protein-protein interaction.

### SUMMARY

The present disclosure provides in embodiments a method for predicting protein-protein interaction, an electronic device and a non-transitory computer readable storage medium.

In an aspect, the present disclosure provides in an embodiment a method for predicting protein-protein interaction, the method including: acquiring a plurality of proteins to be treated, and an amino acid sequence, function information and structure information corresponding to individual proteins; obtaining a fusion representation vector corresponding to the individual proteins based on the amino acid sequence, the function information and the structure information corresponding to the individual proteins by a pre-trained protein representation model; and inputting the fusion representation vector corresponding to the individual proteins to a protein-protein interaction prediction model, to predict the protein-protein interaction.

According to an embodiment of the present disclosure, the method for predicting protein-protein interaction fusion-represents the amino acid sequence, the function information and the structure information corresponding to individual proteins by the pre-trained protein representation model, to obtain a fusion representation vector corresponding to the individual proteins; and inputs the fusion representation vector corresponding to the individual proteins to the protein-protein interaction prediction model, to predict the protein-protein interaction, so that the protein-protein interaction prediction model exhibits better prediction accuracy, robustness and generalization on the basis of accurate fusion representation vector of the protein.

In another aspect, the present disclosure provides in an embodiment an electronic device including: at least one processor; and a memory connected in communication with said at least one processor, wherein the memory stores therein an instruction executable by said at least one processor, and the instruction, that is executed by said at least one processor, implements a method for predicting protein-protein interaction, including: acquiring a plurality of proteins to be treated, and an amino acid sequence, function information and structure information corresponding to individual proteins; obtaining a fusion representation vector corresponding to the individual proteins based on the amino acid sequence, the function information and the structure information corresponding to the individual proteins by a pre-trained protein representation model; and inputting the fusion representation vector corresponding to the individual proteins to a protein-protein interaction prediction model, to predict the protein-protein interaction.

In still another aspect, the present disclosure provides in an embodiment a non-transitory computer readable storage medium having stored therein a computer instruction, wherein the computer instruction causes the computer to implement a method for predicting protein-protein interaction, including: acquiring a plurality of proteins to be treated, and an amino acid sequence, function information and structure information corresponding to individual proteins; obtaining a fusion representation vector corresponding to the individual proteins based on the amino acid sequence, the function information and the structure information corresponding to the individual proteins by a pre-trained protein representation model; and inputting the fusion representation vector corresponding to the individual proteins to a protein-protein interaction prediction model, to predict the protein-protein interaction.

It should be understood that the content described in this section is not intended to identify a key or critical feature of embodiments of the present disclosure, nor is intended to limit the scope of the present disclosure. Other features of the present disclosure will become readily understood from the following description.

### BRIEF DESCRIPTION OF THE DRAWINGS

The accompanying drawings are used to better understand the present disclosure, and do not constitute limitation to the present disclosure.

FIG. 1 is a flow chart showing a method for pre-training a protein representation model according to a first embodiment of the present disclosure.

FIG. 2 is a flow chart showing a method for pre-training a protein representation model according to a second embodiment of the present disclosure.

FIG. 3 is a flow chart showing a method for pre-training a protein representation model according to a third embodiment of the present disclosure.

FIG. 4 is a flow chart showing a method for pre-training a protein representation model according to a fourth embodiment of the present disclosure.

FIG. 5 is a schematic diagram showing a multimodal “sequence-structure-function” protein representation model according to a fifth embodiment of the present disclosure.

FIG. 6 is a flow chart showing a method for predicting protein-protein interaction according to a sixth embodiment of the present disclosure.

FIG. 7 is a block diagram showing an apparatus for pre-training a protein representation model according to a seventh embodiment of the present disclosure.

FIG. 8 is a block diagram showing an apparatus for pre-training a protein representation model according to an eighth embodiment of the present disclosure.

FIG. 9 is a block diagram showing an apparatus for predicting protein-protein interaction according to a ninth embodiment of the present disclosure.

FIG. 10 is a block diagram showing an electronic device for implementing an embodiment of the present disclosure.

### DETAILED DESCRIPTION

Reference will be made to illustrative embodiments of the present disclosure below in combination with the accompanying drawings, which include various details of the embodiments of the present disclosure to facilitate understanding, and should be construed as illustration. Accordingly, those of ordinary skilled in the art will recognize that various changes and modifications can be made to the embodiments described herein without departing from the scope and spirit of the present disclosure. Besides, descriptions on well-known functions and configurations are omitted from the following description for clarity and conciseness.

At present, the method for predicting protein-protein interaction includes two stages: (1) representation of a protein, i.e., representing an amino acid sequence or structure file of a protein in a form of a feature vector that can be understood by a computer model; and (2) a downstream prediction network, i.e., predicting whether protein-protein interaction occurs by a classification or regression model, or predicting an affinity fraction for protein-protein interaction. In general, accuracy of representing a protein is of a great importance for predicting protein-protein interaction by the downstream prediction network. In the related art, a protein representation model is usually pre-trained using protein-based amino acid information, and the protein is then represented on a basis of the pre-trained protein representation model. However, the approach of the protein-based pre-training language model misses a high-level feature such as protein structure and function, which are particularly important for predicting the protein-protein interaction.

For this, the present disclosure provides in embodiments a method for pre-training a protein representation model, which trains a multimodal protein representation model using three-modality synergistic data of protein sequence, structure and function, thereby establishing a better protein representation model.

Reference will be made to a method and apparatus for pre-training a protein representation model, and a method and apparatus for predicting protein-protein interaction below in embodiments of the present disclosure in combination with the accompanying drawings.

FIG. 1 is a flow chart showing a method for pre-training a protein representation model in a first embodiment of the present disclosure.

As shown in FIG. 1, the method for pre-training a protein representation model may include steps 101 to 102.

At the step 101, an amino acid sequence, function information and structure information of a protein are acquired.

It should be noted that an executing main body for a method for pre-training a protein representation model in embodiments of the present disclosure is an apparatus for pre-training a protein representation model, which may be implemented by software and/or hardware and arranged in an electronic device. The electronic device may include but not limited to a terminal device, a server, and etc., which is not particularly limited in any embodiments herein.

In some embodiments, the above function information is textual description information for a protein function

In some embodiments, in order to enable the protein representation model to represent a protein based on structure information useful for protein-protein interaction, the above structure information may be extracted from a structure file corresponding to the protein. In specific, a structure file for the protein may be acquired; point cloud composed of heavy atoms of the protein may be then extracted from the structure file; barcode information of a topological complex of the protein is determined according to the point cloud; and the barcode information is discretized, to obtain the structure information of the protein, thereby obtaining refined structure information of the protein at an atomic level.

The above heavy atom of the protein may include but not limited to a heavy atom such as Carbon (C), Nitrogen (N) and Oxygen (O).

At the step 102, the protein representation model is pre-trained based on the amino acid sequence, the function information and the structure information.

In some embodiments, the pre-training may be performed based on a multimodal “sequence-structure-function” protein representation model.

In some embodiments, in different application scenarios, said pre-training the protein representation model based on the amino acid sequence, the function information and the structure information is implemented by different embodiments. One illustrative embodiment may be inputting the amino acid sequence, the function information and the structure information to the protein representation model to obtain a fusion representation vector; determining a predicted protein corresponding to the fusion representation vector according to a preset decoding network; and pre-training the protein representation model based on the protein and the predicted protein.

In specific, the amino acid sequence, the function information and the structure information may be vectorized to obtain individual vector representations corresponding to the above three information; summing the individual vector representations corresponding to the above three information; and inputting a summed vector representation to the protein representation model, to obtain the fusion representation vector.

It should be noted that the protein representation model may be pre-trained for multiple times, where multiple sets of the amino acid sequence, the function information and the structure information of the protein may be taken as the input, and the number of pre-training times and the number of input sets are not particularly limited herein.

In some embodiments, in order to improve accuracy of the above protein representation model, the above preset decoding network may be classified in accordance with a type of the input protein, where different types of proteins may correspond to different preset decoding networks.

According to embodiments of the present disclosure, the method for pre-training a protein representation model acquires the amino acid sequence, the function information and the structure information of the protein; and pre-trains the protein representation model based on the amino acid sequence, the function information and the structure information, thereby providing a way for pre-training the protein representation model, thus allowing the pre-trained protein representation model to be accurate.

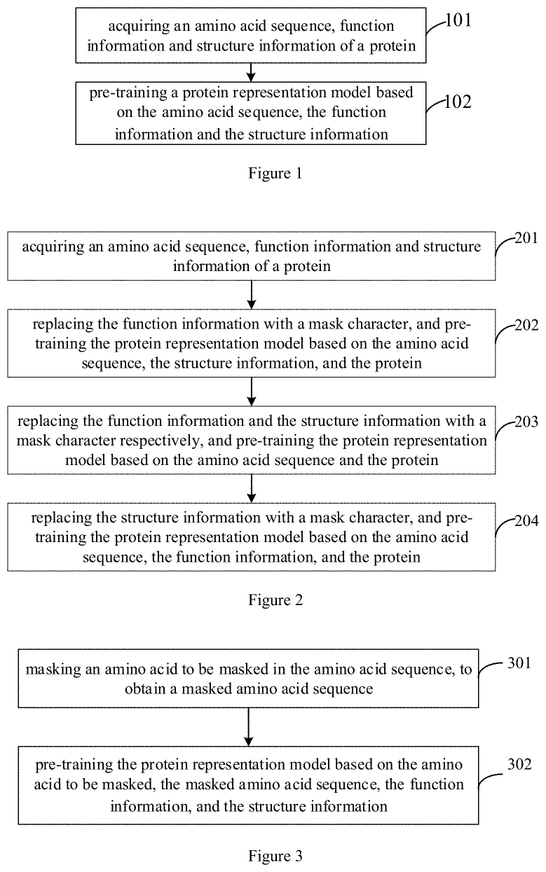

Based on the above embodiments, reference will be further made below in combination with FIG. 2 to illustrate the method in this embodiment.

As shown in FIG. 2, the method may include steps 201 to 204.

At the step 201, an amino acid sequence, function information and structure information of a protein are acquired.

It should be noted that specific implementation for the step 201 may refer to the related description in the above embodiment, which is not repeated here.

At the step 202, the function information is replaced with a mask character, and the protein representation model is pre-trained based on the amino acid sequence, the structure information and the protein.

In some embodiments, an illustrative embodiment for replacing the function information with a mask character and pre-training the protein representation model based on the amino acid sequence, the structure information and the protein may be inputting the amino acid sequence and the structure information to the protein representation model, to obtain a fusion representation vector; inputting the fusion representation vector to a preset decoding network, to obtain a corresponding predicted protein; and adjusting a parameter of the protein representation model according to a difference between the protein and the predicted protein, until the predicted protein is identical to the protein, indicating the protein representation model has been trained successfully.

In this embodiment, in a case of lacking the function information of the protein, in order to enable the protein representation model to accurately represent the protein based on the amino acid sequence and the structure information, in this embodiment, in the process of pre-training the protein representation model, the protein representation model is also pre-trained based on the amino acid sequence and the structure information.

At the step 203, the function information and the structure information are replace with a mask character respectively; and the protein representation model is pre-trained based on the amino acid sequence and the protein.

In some embodiments, in a case of lacking the function information and the structure information of the protein, in order to enable the protein representation model to accurately represent the protein based on the amino acid sequence of the protein, in this embodiment, in the process of pre-training the protein representation model, the protein representation model is also pre-trained based on the amino acid sequence and the protein.

In some embodiments, an illustrative embodiment for pre-training the protein representation model based on the amino acid sequence and the protein may be inputting the amino acid sequence to the protein representation model, to obtain a fusion representation vector; inputting the fusion representation vector to a preset decoding network, to obtain a predicted protein; and pre-training the protein representation model according to a difference between the predicted protein and the protein.

At the step 204, the structure information is replaced with a mask character; and the protein representation model is pre-trained based on the amino acid sequence, the function information and the protein.

In some embodiments, in a case of lacking the structure information of the protein, in order to enable the protein representation model to accurately represent the protein based on the amino acid sequence and the function information of the protein, in this embodiment, in the process of pre-training the protein representation model, the protein representation model is also pre-trained based on the amino acid sequence and the function information of the protein and the protein. In some embodiments, an illustrative embodiment for pre-training the protein representation model based on the amino acid sequence, the function information and the protein may be inputting the amino acid sequence and the function information to the protein representation model, to obtain a fusion representation vector; inputting the fusion representation vector to a preset decoding network, to obtain a predicted protein; and pre-training the protein representation model according to a difference between the predicted protein and the protein.

It should be noted that the protein representation model may be pre-trained based on any one of the above steps 202 to 204 or a combination thereof, which is not particularly limited in this embodiment here.

In some embodiments, in a case where a wrong amino acid present in the amino acid sequence of the protein or in a case of lacking an amino acid in the amino acid sequence of the protein, in order to further improve the accuracy of representing a protein by the protein representation model, based on any one of the above embodiments, as shown in FIG. 3, the method may further include steps 301 and 302.

At the step 301, an amino acid to be masked in the amino acid sequence is masked to obtain a masked amino acid sequence.

In different application scenarios, said masking an amino acid to be masked in the amino acid sequence to obtain a masked amino acid sequence may be implemented by various embodiments, which are illustrated as below.

As an illustrative embodiment, an amino acid to be masked in the amino acid sequence may be replaced with a random character, to obtain a masked amino acid sequence.

As another illustrative embodiment, an amino acid to be masked in the amino acid sequence is replaced with a preset identifier, to obtain a masked amino acid sequence.

At the step 302, the protein representation model is pre-trained based on the amino acid to be masked, the masked amino acid sequence, the function information and the structure information.

In other words, in this embodiment, the protein representation model may also be pre-trained in a way based on a self-supervised mask sequence modeling task.

In some embodiments, in order to accurately pre-train the protein representation model, the above illustrative embodiments for pre-training the protein representation model based on the amino acid to be masked, the masked amino acid sequence, the function information and the structure information may be inputting the masked amino acid sequence, the function information and the structure information to the protein representation model, to obtain a first fusion representation vector; determining an amino acid predicting result corresponding to the amino acid to be masked based on the first fusion representation vector; and pre-training the protein representation model based on the amino acid to be masked and the amino acid predicting result.

In specific, a parameter of the protein representation model is adjusted according to difference information between the amino acid to be masked and the amino acid predicting result, until the difference information between the amino acid to be masked and the amino acid predicting result is less than a preset threshold, or the amino acid to be masked is identical to the amino acid predicting result.

In some embodiments, in order to enable the protein representation model to accurately represent a protein, an illustrative embodiment for inputting the masked amino acid sequence, the function information and the structure information to the protein representation model to obtain a first fusion representation vector is determining a character vector and a position vector corresponding to individual characters in the masked amino acid sequence, the structure information and masked function information, respectively; combining the character vector and the position vector corresponding to the individual characters in the masked amino acid sequence, the structure information and the masked function information, to obtain a combined vector corresponding to the individual characters; and inputting the combined vector corresponding to the individual characters to the protein representation model, to obtain the first fusion representation vector.

It would be understood that the position vector corresponding to a corresponding character in the masked amino acid sequence is to represent a position of the corresponding character (i.e., an amino acid) in the amino acid sequence.

The position vector corresponding to the corresponding character in the function information is to represent a position of the corresponding character in the function information.

The position vector corresponding to the corresponding character in the structure information is always zero.

In specific, based on the multimodal “sequence-structure-function” protein pre-training model, independent Position Embedding is introduced for two sequenced modalities of protein sequence and protein function, so that the model can obtain order information of the amino acids and the functional description text. The individual characters in the masked amino acid sequence, the structure information and the masked function information correspond to a character vector and position vector having a feature vector. The character vector and the position vector corresponding to the individual characters in the masked amino acid sequence, the structure information and the masked function information are combined, thereby obtaining a combined vector corresponding to the individual characters. The combined vector corresponding to the individual characters is input to the protein representation model, to obtain the first fusion representation vector.

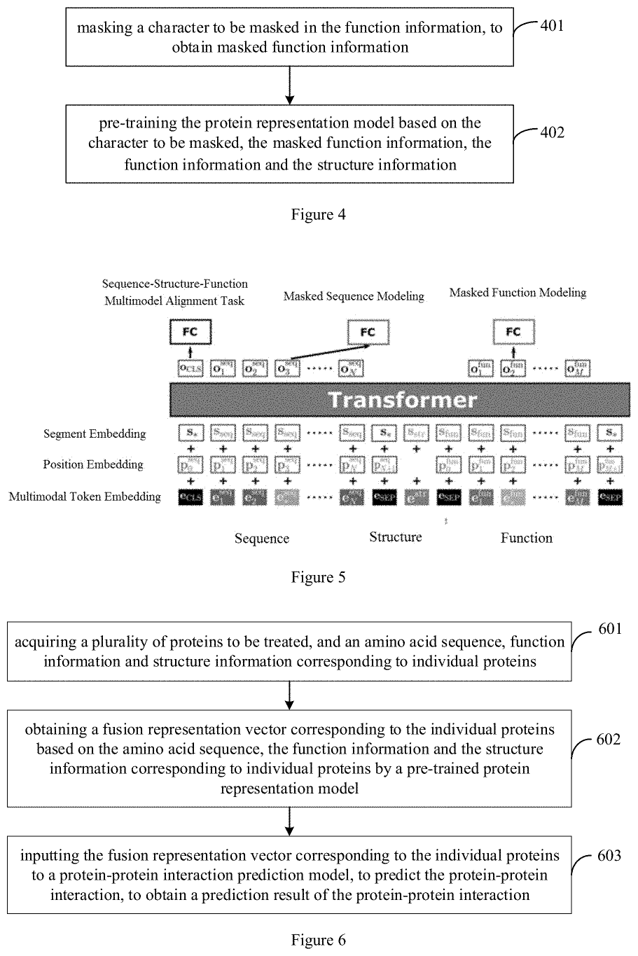

In some embodiments, in a case of where a wrong character is present in the function information of the protein or in a case of lacking a character in the function information of the protein, in order to further improve accuracy of representing the protein by the protein representation model to enable the trained protein representation model to accurately represent the protein based on absence of a character or presence of a wrong character in the function information, on the basis of any one of the above embodiments, as shown in FIG. 4, the method for pre-training a protein representation model may further include steps 401 to 402.

At the step 401, a character to be masked in the function information is masked to obtain masked function information.

In different application scenarios, said masking a character to be masked in the function information to obtain masked function information may be implemented by various embodiments, which are illustrated as below.

As an illustrative embodiment, a character to be masked in the function information may be replaced with a random character, to obtain masked function information.

As an illustrative embodiment, a character to be masked in the function information may be replaced with a preset identifier, to obtain masked function information.

At the step 402, the protein representation model is pre-trained based on the character to be masked, the masked function information, the function information and the structure information.

In other words, in this embodiment, the protein representation model may also be pre-trained in a way based on a self-supervised mask function modeling task.

In some embodiments, in order to accurately pre-train the protein representation model, an illustrative embodiment for pre-training the protein representation model based on the character to be masked, the masked function information, the function information and the structure information is inputting the masked function information, the function information and the structure information to the protein representation model, to obtain a second fusion representation vector; determining a character predicting result corresponding to the character to be masked based on the second fusion representation vector; and pre-training the protein representation model based on the character to be masked and the character predicting result.

In specific, a parameter of the protein representation model is adjusted according to difference information between the character to be masked and the character predicting result, until the character to be masked is identical to the character predicting result; the pre-training of the protein representation model is ended.

In some embodiments, in order for those skilled in the art to clearly understand the present disclosure, reference will be made below to illustratively describe the method for pre-training a protein representation model in an embodiment in combination with FIG. 5.

In should be noted that this embodiment is implemented on a basis of a multimodal “sequence-structure-function” protein pre-training model, which is a Transformer-based single-stream multimodal pre-training model, where different modalities are distinguished by Segment Embedding. Unlike a Transformer-based single-modal model having only one set of Position Embedding, the multimodal pre-training model introduces independent Position Embedding for the two sequenced modalities of protein sequence and protein function (i.e., the textual description for the protein function), so that the multimodal pre-training model is provided with the order information of the amino acid and functional description text. The Multimodal Token Embedding contains 3 modalities of sequence, structure and function. The multimodal pre-training model introduces a self-supervised Masked Sequence Modeling task and a Masked Function Modeling task for sequenced protein data of amino acid sequence and functional description. Besides, for learning synergistic information among multiple modalities, a multimodal “sequence-structure-function” alignment task is introduced in an embodiment of the present disclosure. In the process of pre-training the protein representation model by the multimodal alignment task, reference may be made to related description in the embodiment for FIG. 2, which is not repeated here.

The present disclosure further provides in an embodiment a method for predicting protein-protein interaction.

FIG. 6 is a flow chart showing a method for predicting protein-protein interaction according to a sixth embodiment of the present disclosure.

As shown in FIG. 6, the method for predicting protein-protein interaction may include steps 601 to 603.

At the step 601, a plurality of proteins to be treated, and an amino acid sequence, function information and structure information corresponding to individual proteins are acquired.

At the step 602, a fusion representation vector corresponding to the individual proteins is obtained based on the amino acid sequence, the function information and the structure information corresponding to the individual proteins by a protein representation model pre-trained according to the method as described in any one of the above embodiments.

In specific, a fusion representation vector corresponding to the individual proteins is obtained by taking protein pre-training representation as an input and on the basis of the pre-trained protein representation model.

The specific process of pre-training a protein representation model may refer to related description in the above embodiments, which is not repeated here

At the step 603, the fusion representation vector corresponding to the individual proteins is input to a protein-protein interaction prediction model, to predict the protein-protein interaction, to obtain a prediction result of the protein-protein interaction.

In specific, for a downstream neural network under different protein-protein interaction tasks, the fusion representation vector corresponding to the individual proteins is taken as the input for predicting the protein-protein interaction, to obtain a prediction result of the protein-protein interaction.

It should be noted that the protein-protein interaction prediction model can be designed as various downstream task networks to meet needs of different types of proteins. For example, the downstream task model may take one pair of proteins as the input for the protein-protein interaction task, or take three proteins as the input for the protein-protein interaction task, or take two pairs of proteins as the input for the protein-protein interaction task.

According to an embodiment of the present disclosure, the method for predicting protein-protein interaction fusion-represents the amino acid sequence, the function information and the structure information corresponding to individual proteins by the pre-trained protein representation model, to obtain the fusion representation vector corresponding to the individual proteins; and inputs the fusion representation vector corresponding to the individual proteins to the protein-protein interaction prediction model, to predict the protein-protein interaction, to obtain a prediction result of the protein-protein interaction, so that the protein-protein interaction prediction model exhibits better prediction accuracy, robustness and generalization on the basis of accurate fusion representation vector of the protein.

To implement the above embodiments, the present disclosure further provides in an embodiment an apparatus for pre-training a protein representation model.

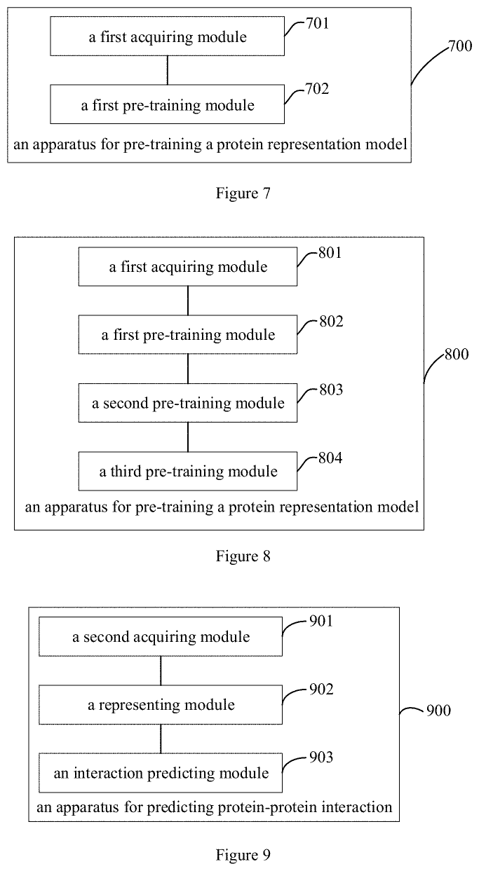

FIG. 7 is a block diagram showing an apparatus for pre-training a protein representation model according to a seventh embodiment of the present disclosure.

As shown in FIG. 7, the apparatus 700 for pre-training a protein representation model may include a first acquiring module 701 and a first pre-training module 702.

The first acquiring module 701 is configured to acquire an amino acid sequence, function information and structure information of a protein.

The first pre-training module 702 is configured to pre-train the protein representation model based on the amino acid sequence, the function information and the structure information.

According to an embodiment of the present disclosure, the apparatus for pre-training a protein representation model acquires an amino acid sequence, function information and structure information of a protein; and pre-trains the protein representation model based on the amino acid sequence, the function information and the structure information, thereby providing a way for pre-training the protein representation model, thus allowing the pre-trained protein representation model to be accurate.

In some embodiments, as shown in FIG. 8, the apparatus 800 for pre-training a protein representation model may include a first acquiring module 801, a first pre-training module 802, a second pre-training module 803 and a third pre-training module 804.

It should be noted that detailed description for the first acquiring module 801 may refer to illustration on the first acquiring module 701 in the embodiment as shown in FIG. 7, which is not repeated here.

In some embodiments, the above first pre-training module 802 is specifically configured to replace the function information with a mask character and pre-train the protein representation model based on the amino acid sequence, the structure information and the protein; and/or to replace the function information and the structure information with a mask character respectively and pre-train the protein representation model based on the amino acid sequence and the protein; and/or to replace the structure information with a mask character and pre-train the protein representation model based on the amino acid sequence, the function information and the protein.

In some embodiments, the second pre-training module 803 is configured to mask an amino acid to be masked in the amino acid sequence, to obtain a masked amino acid sequence; and to pre-train the protein representation model based on the amino acid to be masked, the masked amino acid sequence, the function information and the structure information.

In some embodiments, the second pre-training module 803 is specifically configured to input the masked amino acid sequence, the function information and the structure information to the protein representation model, to obtain a first fusion representation vector; to determine an amino acid predicting result corresponding to the amino acid to be masked based on the first fusion representation vector; and to pre-train the protein representation model based on the amino acid to be masked and the amino acid predicting result. In some embodiments, the above illustrative embodiment for inputting the masked amino acid sequence, the function information and the structure information to the protein representation model to obtain a first fusion representation vector is determining a character vector and a position vector corresponding to individual characters in the masked amino acid sequence, the structure information and masked function information, respectively; combining the character vector and the position vector corresponding to the individual characters in the masked amino acid sequence, the structure information and the masked function information, to obtain a combined vector corresponding to the individual characters; and inputting the combined vector corresponding to the individual characters to the protein representation model, to obtain the first fusion representation vector.

In some embodiments, the third pre-training module 804 is configured to mask a character to be masked in the function information, to obtain masked function information; and to pre-train the protein representation model based on the character to be masked, the masked function information, the function information and the structure information.

In some embodiments of the present disclosure, the above third pre-training module 804 is specifically configured to input the masked function information, the function information and the structure information to the protein representation model, to obtain a second fusion representation vector; to determine a character predicting result corresponding to the character to be masked based on the second fusion representation vector; and to pre-train the protein representation model based on the character to be masked and the character predicting result. In some embodiments, the structure information is obtained by: acquiring a structure file for the protein; extracting point cloud composed of heavy atoms of the protein from the structure file; determining barcode information of a topological complex of the protein according to the point cloud; and discretizing the barcode information, to obtain the structure information of the protein.

It should be noted that explanation and illustration on the method for pre-training a protein representation model in the above embodiments are also applicable to the apparatus for pre-training a protein representation model in this embodiment, which are not repeated here.

The present disclosure further provides in an embodiment an apparatus for predicting protein-protein interaction.

FIG. 9 is a block diagram showing an apparatus for predicting protein-protein interaction according to a ninth embodiment of the present disclosure.

As shown in FIG. 9, the apparatus 900 for pre-training protein-protein interaction may include a second acquiring module 901, a representing module 902, and an interaction predicting module 903.

The second acquiring module 901 is configured to acquire a plurality of proteins to be treated, and an amino acid sequence, function information and structure information corresponding to individual proteins.

The representing module 902 is configured to obtain a fusion representation vector corresponding to the individual proteins based on the amino acid sequence, the function information and the structure information corresponding to the individual proteins by a protein representation model pre-trained according to the method as described in any one of the above embodiments.

The interaction predicting module 903 is configured to input the fusion representation vector corresponding to the individual proteins to a protein-protein interaction prediction model, to predict the protein-protein interaction, to obtain a prediction result of the protein-protein interaction.

It should be noted that explanation and illustration on the method for predicting protein-protein interaction in the above embodiments are also applicable to the apparatus for predicting protein-protein interaction in this embodiment, which are not repeated here.

According to an embodiment of the present disclosure, the apparatus for predicting protein-protein interaction fusion-represents the amino acid sequence, the function information and the structure information corresponding to individual proteins by the pre-trained protein representation model, to obtain a fusion representation vector corresponding to the individual proteins; and inputs the fusion representation vector corresponding to the individual proteins to the protein-protein interaction prediction model, to predict the protein-protein interaction, to obtain a prediction result of the protein-protein interaction, so that the protein-protein interaction prediction model exhibits better prediction accuracy, robustness and generalization on the basis of accurate fusion representation vector of the protein.

According to embodiments of the present disclosure, the present disclosure further provides in an embodiment an electronic device, a readable storage medium and a computer program product.

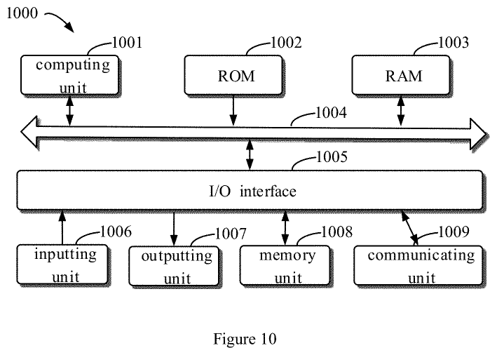

FIG. 10 is a block diagram showing an electronic device 1000 for implementing an embodiment of the present disclosure. The electronic device is intended to represent various forms of digital computers, such as a laptop, a desktop, a workstation, a personal digital assistant, a server, a blade server, a mainframe computer, and other suitable computers. The electronic device may also represent various forms of mobile devices, such as a personal digital processor, a cellular phone, a smart phone, a wearable device, and other similar computing devices. The components shown herein, their connections and relationships, and their functions are by way of examples only, and are not intended to limit implementations of the present disclosure described and/or claimed herein.

As shown in FIG. 10, the device 1000 includes a computing unit 1001 to perform various appropriate actions and processes according to a computer program stored in a read only memory (ROM) 1002 or a computer program loaded from a storage unit 1008 into a random access memory (RAM) 1003. The RAM 1003 may also stores therein various programs and data required for the operation of the storage device 1000. The computing unit 1001, the ROM 1002, and the RAM 1003 are connected to each other through a bus 1004. An input/output (I/O) interface 1005 is also connected to the bus 1004.

Components in the device 1000, connected to the I/O interface 1005, includes: an input unit 1006, such as a keyboard and a mouse; an output unit 1007, such as various types of displays and speakers; a storage unit 1008, such as a disk and an optical disk; and a communication unit 1009, such as a network card, a modem, and a wireless communication transceiver. The communication unit 1009 allows the device 1000 to exchange information/data with other devices through a computer network such as the Internet and/or various telecommunication networks.

The computing unit 1001 may be various general-purpose and/or special-purpose processing components with processing and computing capabilities. Some examples of the computing unit 1001 include, but are not limited to, a central processing unit (CPU), a graphic processing unit (GPU), various specialized artificial intelligence (AI) computing chips, various computing units that run machine learning model algorithms, a digital signal processor (DSP), and any suitable processor, controller, microcontroller, etc. The computing unit 1001 performs various methods and processes described above, such as a method for pre-training a protein representation model. For example, in some embodiments, the method for pre-training a protein representation model may be implemented as a computer software program that is tangibly embodied on a machine-readable medium, such as the storage unit 1008. In some embodiments, a part or all of the computer program may be loaded and/or installed on the device 1000 via the ROM 1002 and/or the communication unit 1009. When the computer program is loaded into the RAM 1003 and executed by the computing unit 1001, one or more steps of the method for pre-training a protein representation model described above may be performed. Alternatively, in other embodiments, the computing unit 1001 may be configured to perform the method for pre-training a protein representation model in any other suitable manner (e.g., by means of firmware).

In other embodiments, the above-mentioned computing unit 1001 performs the method for predicting protein-protein interaction described above. For example, in some embodiments, the method for predicting protein-protein interaction can be implemented as a computer software program, which is tangible embodied in a machine-readable medium, such as storage unit 1008. In some embodiments, a part or all of the computer program may be loaded and/or installed on the device 1000 via the ROM 1002 and/or the communication unit 1009. When the computer program is loaded into RAM 1003 and executed by computing unit 1001, one or more steps of the method for predicting protein-protein interaction described above may be performed. Alternatively, in other embodiments, the computing unit 1001 may be configured to perform the method for predicting protein-protein interaction by any other suitable means (e.g., by means of firmware).

Various implementations of the system and technique described herein above may be implemented in a digital electronic circuit, an integrated circuit system, a field programmable gate array (FPGA), an application specific integrated circuit (ASIC), an application specific standard product (ASSP), a system-on-chip (SOC), a complex programmable logic device (CPLD), a computer hardware, a firmware, a software, and/or a combination thereof. These various embodiments may include implementation in one or more computer programs executable and/or interpretable on a programmable system including at least one programmable processor. The programmable processor may be a special-purpose or general-purpose programmable processor, which may receive data and instructions from a storage system, at least one input device and at least one output device, and may transmit data and instructions to the storage system, the at least one input device, and the at least one output device.

A program code for implementing the methods of the present disclosure may be written in any combination of one or more programming languages. The program code may be provided to a processor or controller of a general computer, a dedicated computer, or other programmable data processing devices, such that the program code, when executed by the processor or controller, causes the functions and/or operations specified in the flow chart and/or the block diagram are(is) performed. The program code can be executed entirely on the machine, partly on the machine, as a stand-alone software package partly on a machine and partly on a remote machine or entirely on a remote machine or server.

In the context of the present disclosure, a machine-readable medium may be a tangible medium that may contain or store a program for use by or in connection with an instruction execution system, apparatus, or device. The machine-readable medium may be a machine-readable signal medium or a machine-readable storage medium. A machine-readable medium may include, but is not limited to, an electronic, magnetic, optical, electromagnetic, infrared, or semiconductor system, apparatus, or device, or any suitable combination of the foregoing. More specific examples of the machine-readable storage medium include an electrical connection based on one or more wires, a portable computer disk, a hard disk, a random access memory (RAM), a read-only memory (ROM), an erasable programmable read-only memory (EPROM or flash memory), a fiber optics, compact disc read-only memory (CD-ROM), an optical storage device, a magnetic storage device, or any suitable combination of the foregoing.

To provide interaction with a user, the system and technique described herein may be implemented on a computer having a display device (e.g., a cathode ray tube (CRT) or a liquid crystal display (LCD)) for displaying information for the user; and a keyboard and a pointing device (e.g., a mouse or a trackball) through which a user can provide an input to the computer. Other types of devices can also be used to provide interaction with the user, for example, the feedback provided to the user may be any form of sensory feedback (e.g., visual feedback, auditory feedback, or tactile feedback); and may be in any form (including acoustic input, voice input, or tactile input) to receive the input from the user.

The system and technique described herein may be implemented on a computing system that includes a back-end component (e.g., a data server), a computing system that includes a middleware component (e.g., an application server), a computing system that includes a front-end component (e.g., a user computer with a graphical user interface or a web browser through which a user can interact with implementations of the system and technique described herein), or a computing system that includes said backend component, said middleware component, and said front-end component or any combination thereof. The components of the system may be interconnected by any form or medium of digital data communication (e.g., a communication network). Examples of the communication network include: a Local Area Network (LAN), a Wide Area Network (WAN), an Internet and a blockchain network.

The computer system may include a client and a server. The client and server are generally remote from each other and usually interact through a communication network. A relationship between the client and the server is generated by a computer program running on a corresponding computer and having a client-server relationship with each other. The server may be a cloud server, also known as a cloud computing server or a cloud host, which is a host product in the cloud computing service system, and solves the defects of difficult management and weak business expansion in a traditional physical host and a virtual private server (“VPS” for short). The server may be a cloud server, a server of a distributed system, or a server combined with a blockchain.

It should be noted that artificial intelligence (AI) is the study of making a computer to simulate certain thinking processes and intelligent behaviors of people (such as learning, reasoning, thinking, and planning), including both hardware-level technology and software-level technology. The AI hardware technology generally includes technologies such as a sensor, a dedicated AI chip, cloud computing, distributed storage, and big data processing. The AI software technology mainly includes computer vision technology, speech recognition technology, natural language processing technology, and machine learning/depth learning, big data processing technology, knowledge graph technology and other major directions

It should be understood that the steps may be reordered, added or deleted by using the various forms of flows shown above. For example, the steps described in the present disclosure may be executed in parallel, sequentially, or in different orders, as long as the desired results of the technical solutions disclosed in the present disclosure can be achieved, and no limitation is imposed herein.

The above-mentioned specific embodiments do not limit the protection scope of the present disclosure. It should be understood by those skilled in the art that various modifications, combinations, sub-combinations and replacements may be made depending on design requirements and other factors. Any modifications, equivalent replacements, and improvements made within the spirit and principles of the present disclosure should be included within the protection scope of the present disclosure.

What is claimed is:

1. A method for predicting protein-protein interaction, comprising:
acquiring a plurality of proteins to be treated, and an amino acid sequence, function information and structure information corresponding to individual proteins;
obtaining a fusion representation vector corresponding to the individual proteins based on the amino acid sequence, the function information and the structure information corresponding to the individual proteins by a pre-trained protein representation model; and
inputting the fusion representation vector corresponding to the individual proteins to a protein-protein interaction prediction model, to predict the protein-protein interaction.

2. The method according to claim 1, wherein the pre-trained protein representation model is obtained by:
acquiring an amino acid sequence, function information and structure information of a protein; and
pre-training the protein representation model based on the amino acid sequence, the function information and the structure information.

3. The method according to claim 2, wherein pre-training the protein representation model based on the amino acid sequence, the function information and the structure information comprises one or more of:
replacing the function information with a mask character, and pre-training the protein representation model based on the amino acid sequence, the structure information and the protein;
replacing the function information and the structure information with a mask character respectively, and pre-training the protein representation model based on the amino acid sequence and the protein; and
replacing the structure information with a mask character, and pre-training the protein representation model based on the amino acid sequence, the function information and the protein.

4. The method according to claim 3, wherein the pre-trained protein representation model is obtained further by
masking an amino acid to be masked in the amino acid sequence, to obtain a masked amino acid sequence; and
pre-training the protein representation model based on the amino acid to be masked, the masked amino acid sequence, the function information and the structure information.

5. The method according to claim 4, wherein pre-training the protein representation model based on the amino acid to be masked, the masked amino acid sequence, the function information and the structure information comprises:
inputting the masked amino acid sequence, the function information and the structure information to the protein representation model, to obtain a first fusion representation vector;
determining an amino acid predicting result corresponding to the amino acid to be masked based on the first fusion representation vector; and
pre-training the protein representation model based on the amino acid to be masked and the amino acid predicting result.

6. The method according to claim 5, wherein inputting the masked amino acid sequence, the function information and the structure information to the protein representation model, to obtain a first fusion representation vector comprises:
determining a character vector and a position vector corresponding to individual characters in the masked amino acid, the structure information and masked function information, respectively;
combining the character vector and the position vector corresponding to the individual characters in the masked amino acid, the structure information and the masked function information, to obtain a combined vector corresponding to the individual characters; and
inputting the combined vector corresponding to the individual characters to the protein representation model, to obtain the first fusion representation vector.

7. The method according to claim 3, wherein the pre-trained protein representation model is obtained further by:
masking a character to be masked in the function information, to obtain masked function information; and
pre-training the protein representation model based on the character to be masked, the masked function information, the function information and the structure information.

8. The method according to claim 7, wherein pre-training the protein representation model based on the character to be masked, the masked function information, the function information and the structure information comprises:
inputting the masked function information, the function information and the structure information to the protein representation model, to obtain a second fusion representation vector;
determining a character predicting result corresponding to the character to be masked based on the second fusion representation vector; and
pre-training the protein representation model based on the character to be masked and the character predicting result.

9. The method according to claim 2, wherein the structure information is obtained by:
acquiring a structure file for the protein;
extracting point cloud composed of heavy atoms of the protein from the structure file;
determining barcode information of a topological complex of the protein according to the point cloud; and
discretizing the barcode information, to obtain the structure information of the protein.

10. An electronic device, comprising:
at least one processor; and
a memory connected in communication with said at least one processor, wherein
the memory stores therein an instruction executable by said at least one processor, and
the instruction, that is executed by said at least one processor, implements a method for predicting protein-protein interaction, comprising:
acquiring a plurality of proteins to be treated, and an amino acid sequence, function information and structure information corresponding to individual proteins;
obtaining a fusion representation vector corresponding to the individual proteins based on the amino acid sequence, the function information and the structure information corresponding to the individual proteins by a pre-trained protein representation model; and
inputting the fusion representation vector corresponding to the individual proteins to a protein-protein interaction prediction model, to predict the protein-protein interaction.

11. The electronic device according to claim 10, wherein the pre-trained protein representation model is obtained by:
acquiring an amino acid sequence, function information and structure information of a protein; and
pre-training the protein representation model based on the amino acid sequence, the function information and the structure information.

12. The electronic device according to claim 11, wherein pre-training the protein representation model based on the amino acid sequence, the function information and the structure information comprises one or more of:
replacing the function information with a mask character, and pre-training the protein representation model based on the amino acid sequence, the structure information and the protein;
replacing the function information and the structure information with a mask character respectively, and pre-training the protein representation model based on the amino acid sequence and the protein; and
replacing the structure information with a mask character, and pre-training the protein representation model based on the amino acid sequence, the function information and the protein.

13. The electronic device according to claim 12, wherein the method further comprises:
masking an amino acid to be masked in the amino acid sequence, to obtain a masked amino acid sequence; and
pre-training the protein representation model based on the amino acid to be masked, the masked amino acid sequence, the function information and the structure information.

14. The electronic device according to claim 13, wherein pre-training the protein representation model based on the amino acid to be masked, the masked amino acid sequence, the function information and the structure information comprises:
inputting the masked amino acid sequence, the function information and the structure information to the protein representation model, to obtain a first fusion representation vector;
determining an amino acid predicting result corresponding to the amino acid to be masked based on the first fusion representation vector; and
pre-training the protein representation model based on the amino acid to be masked and the amino acid predicting result.

15. The electronic device according to claim 14, wherein inputting the masked amino acid sequence, the function information and the structure information to the protein representation model, to obtain a first fusion representation vector comprises:
determining a character vector and a position vector corresponding to individual characters in the masked amino acid, the structure information and masked function information, respectively;
combining the character vector and the position vector corresponding to the individual characters in the masked amino acid, the structure information and the masked function information, to obtain a combined vector corresponding to the individual characters; and
inputting the combined vector corresponding to the individual characters to the protein representation model, to obtain the first fusion representation vector.

16. The electronic device according to claim 12, wherein the method further comprises:
masking a character to be masked in the function information, to obtain masked function information; and
pre-training the protein representation model based on the character to be masked, the masked function information, the function information and the structure information.

17. The electronic device according to claim 16, wherein pre-training the protein representation model based on the character to be masked, the masked function information, the function information and the structure information comprises:
inputting the masked function information, the function information and the structure information to the protein representation model, to obtain a second fusion representation vector;
determining a character predicting result corresponding to the character to be masked based on the second fusion representation vector; and
pre-training the protein representation model based on the character to be masked and the character predicting result.

18. The electronic device according to claim 11, wherein the structure information is obtained by:
acquiring a structure file for the protein;
extracting point cloud composed of heavy atoms of the protein from the structure file;
determining barcode information of a topological complex of the protein according to the point cloud; and
discretizing the barcode information, to obtain the structure information of the protein.

19. A non-transitory computer readable storage medium having stored therein a computer instruction, wherein the computer instruction causes the computer to implement a method for predicting protein-protein interaction, comprising:
acquiring a plurality of proteins to be treated, and an amino acid sequence, function information and structure information corresponding to individual proteins;
obtaining a fusion representation vector corresponding to the individual proteins based on the amino acid sequence, the function information and the structure information corresponding to the individual proteins by a pre-trained protein representation model; and
inputting the fusion representation vector corresponding to the individual proteins to a protein-protein interaction prediction model, to predict the protein-protein interaction.
